(19) United States
(12) Patent Application Publication
(10) Pub. No.: US 2024/0109951 A1
(43) Pub. Date: Apr. 4, 2024

(54) METHODS AND COMPOUNDS FOR ELIMINATING IMMUNE RESPONSES TO THERAPEUTIC AGENTS

(71) Applicant: IMCYSE SA, Liège (BE)

(72) Inventors: Jean-Marie SAINT-REMY, Grez-Doiceau (BE); Luc VANDER ELST, Obaix (BE); Vincent CARLIER, Énines (BE)

(21) Appl. No.: 18/462,035

(22) Filed: Sep. 6, 2023

Related U.S. Application Data
(63) Continuation of application No. 15/761,223, filed on Mar. 19, 2018, now Pat. No. 11,787,849.

(30) Foreign Application Priority Data
Sep. 25, 2015 (EP) 15186845.2

(51) Int. Cl. C07K 14/74; A61K 39/00; A61K 39/39; C12N 9/02

(52) U.S. Cl. CPC C07K 14/70539; A61K 39/001; A61K 39/39; C12N 9/0004; A61K 38/00

(57) ABSTRACT

The invention describes kit of parts of polypeptides comprising: a) a peptide comprising: a1) an MHC class II T cell epitope or a CD1 d-restricted NKT cell epitope, and a2) immediately adjacent to said epitope or separated by at most 7 amino acids from said epitope a [CST]-X(2)-C [SEQ ID NO:7] or C-X(2)-[CST] [SEQ ID NO:8] oxidoreductase motif sequence, and b) a poly-peptide comprising: b1) a therapeutic protein and b2) the epitope defined in a1), wherein the epitope sequence is a sequence which differs from the sequence of the protein of b1). The therapeutic protein, in combination with the peptide, is used to prevent an immune response against the therapeutic protein.

### CROSS-REFERENCE TO RELATED APPLICATIONS

This application is a continuation of U.S. application Ser. No. 15/761,223, filed on Mar. 19, 2018, which is a U.S. national stage entry under 35 U.S.C. § 371 of PCT International Patent Application No. PCT/EP2016/072690, filed Sep. 23, 2016, which claims priority to European Patent Application No. 15186845.2, filed Sep. 25, 2015, the contents of each of which are incorporated herein by reference in their entirety.

### REFERENCE TO SEQUENCE LISTING SUBMITTED ELECTRONICALLY

The content of the electronically submitted sequence listing (Name: 2752_0154_Sequence_Listing.xml; Size: 72,715 bytes; and Date of Creation: Sep. 6, 2023) filed with the application is incorporated herein by reference in its entirety.

### FIELD OF THE INVENTION

The present invention relates to modified proteins for use in replacement therapies, gene therapy and gene vaccinations.

The present invention further relates to compounds and methods to prevent an immune response against proteins used in replacement therapies, gene therapy and gene vaccinations.

### BACKGROUND OF THE INVENTION

Proteins which are used as therapeutic agents often elicit an immune response which precludes further use. Examples are Factor VIII in the treatment of haemophilia A patients, and antibodies specific for cytokines (e.g. anti-TNF-alpha antibodies) or specific for cell surface markers (e.g. anti-CD20 antibodies). On average 30% of the patients treated by such reagents develop antibodies at concentrations detectable in peripheral blood. In addition, a significant proportion of patients require administration of higher than expected doses of the therapeutic agents, suggesting that antibodies at concentrations below detection neutralise the activity of the agent and/or increase its clearance from the circulation. In all these situations, it would be advantageous to prevent these immune responses.

Gene therapy and gene vaccination rely on viral vectors used to carry out transgenesis. However, viral proteins expressed by these vectors elicit immune responses, reducing the efficiency of transgenesis and preventing re-administration of the transgene. Avoiding such response would allow long-term expression of the transgene, and would reduce the number of viral particles needed to achieve functional transgenesis or vaccination efficiency.

Patent application WO2008017517 describes peptides with a class II-restricted T cell epitope of an antigen and a redox motif sequence and their use in the therapy of a number of diseases. Further details are published in Carlier et al. (2012), PloS ONE 7, e45366. The use of such peptides results in the generation of CD4+ T cells with cytolytic properties which induce apoptosis of antigen-presenting cells (presenting the antigen used in the design of the peptide) after cognate interaction with peptide-MHC class II complexes.

Further patent applications of the same inventor disclose the use of this technology to avoid an immune response against therapeutic proteins or antibodies (WO2009101206) or against proteins encoded by the backbone of a viral vector for gene therapy of vaccination (WO2009101204).

Promiscuous MHC class II T cell epitopes binding to a plurality of MHC class II molecules have been described for a variety of antigens.

A fusion protein of VEGF and a promiscuous T helper cell epitope is disclosed in US2007184023.

A fusion protein of zona pellucida protein and a tetanus toxin promiscuous T cell epitope generates antibodies which bind to endogenous zona pellucida protein and act as an anticonceptive (Lou et al. (1995) J. Immunol. 155, 2715-2720).

### SUMMARY OF THE INVENTION

The present invention provides solutions for the following problems.

In the prior art methods, described above, the sequence of the epitope which is used in the peptide is defined by the sequence of the antigen.

When isolated and fused to the redox motif sequence, such epitope peptide can be subject to proteolytic cleavage, may be difficult to solubilise, or may tend to form internal cystine bridges with the redox motif.

Further, the use of different peptides for different therapeutic proteins makes it difficult to design standard procedures wherein peptides can be administrated at a same concentration and in a same vaccination scheme regardless of the type of therapeutic protein which is used.

The large polymorphism of MHC class II determinants in humans makes it difficult to use a single or a small number of epitopes from one protein to match this polymorphism.

Only for those antigens that contain a promiscuous epitope, or for those antigens wherein different epitopes of an antigen can be identified to circumvent the polymorphism, it is possible to treat any individual independent of his/her HLA type.

If present in a protein, the use of a CD1d-restricted NKT cell peptide epitope can bypass the HLA polymorphism, however not all antigenic proteins contain such peptide sequence.

The present invention allows uncoupling the connection between an antigen containing a T cell epitope and the use of the same epitope to make a peptide with an epitope and a redox motif sequence.

The present invention allows using T cell epitopes which bind to different MHC class II proteins and alleles.

As can be easily understood from the medical uses as described in the present application, and described in more detail below these medical uses require a first administration of the peptide comprising the epitope and the oxidoreductase motif to elicit a population of cytolytic CD4+ against Antigen Presenting Cells (APC) presenting the epitope. This is followed by a second administration of a therapeutic protein or a vector for gene therapy with the newly introduced epitope. Herein the therapeutic protein (or the viral vector protein encoded by the vector) contains the same epitope as present in the peptide used in the first administration. An eventual immune response to the e.g. a therapeutic protein would start with the presentation of epitopes of the therapeutic protein by APC. The APC will not only present the epitopes present in the therapeutic protein itself but also the epitope which has been introduced. As a consequence the APC will be killed by the above cytolytic cells, preventing the clearance of the antigen.

The medical uses of the present invention are based on the separate administration of the two types of polypeptides. They are accordingly referred to by one of the synonyms “combination”, “set” or “kit of parts”.

An aspect of the invention relates to kits of parts of polypeptides comprising:
a) a peptide comprising
a1) an MHC class II T cell epitope or a CD1d-restricted NKT cell epitope, and
a2) immediately adjacent to said epitope or separated by at most 7 amino acids from said epitope a [CST]-X(2)-C [SEQ ID NO:7] or C-X(2)-[CST][SEQ ID NO:8] oxidoreductase motif sequence, and
b) a polypeptide comprising:
b1) a therapeutic protein and
b2) the epitope defined in a1), wherein the epitope sequence is a sequence which differs from the sequence of the protein of b1).

An aspect of the invention relates to kits of parts of comprising
a) a peptide comprising:
a1) an MHC class II T cell epitope or a CD1d-restricted NKT cell epitope, and
a2) immediately adjacent to said epitope or separated by at most 7 amino acids from said epitope a [CST]-X(2)-C [SEQ ID NO:7] or C-X(2)-[CST][SEQ ID NO:8] oxidoreductase motif sequence,
and
b) an expression vector comprising a polynucleotide sequence encoding a polypeptide comprising:
b1) a therapeutic protein and
b2) the epitope defined in a1), wherein the epitope sequence is a sequence which differs from the sequence of the protein of b1).

An aspect of the invention relates to kits of parts of comprising
a) a peptide comprising:
a1) an MHC class II T cell epitope or a CD1d-restricted NKT cell epitope, and
a2) immediately adjacent to said epitope or separated by at most 7 amino acids from said epitope a [CST]-X(2)-C [SEQ ID NO:7] or C-X(2)-[CST][SEQ ID NO:8] oxidoreductase motif sequence,
and
b) a viral vector for gene therapy or gene vaccination comprising in the backbone a polynucleotide sequence encoding a protein comprising:
b1) a viral vector protein for the function and maintenance of the vector,
b2) the epitope defined in a1), wherein the epitope sequence is a sequence which differs from the sequence of the protein of b1).

Thus the sequence of the epitope, does not occur in the natural (or wild type or native) sequence of the therapeutic protein or the viral vector protein as used in the state of the art therapy. The invention does not envisage fusion proteins wherein an existing epitope within the protein is repeated as a fusion partner of the protein. The invention does not envisage proteins wherein the above defined epitope of a) would be an existing epitope which is excised from the sequence of the protein and inserted at another part of the proteins by deletion and insertion via recombinant DNA technology.

In embodiments of these kits the protein of b) is a fusion protein comprising as fusion partners:
b1) a therapeutic protein or a viral vector protein and
b2) the epitope defined in a1), wherein the epitope sequence is a sequence which differs from the sequence of the protein of b1).

In embodiments of these kits the oxidoreductase motif sequence is C-X(2)-C [SEQ ID NO:2].

In embodiments of these kits CD1d-restricted NKT cell epitope motif has the sequence [FWYHT]-X(2)-[VILM]-X(2)-[FWYHT] [SEQ ID NO:1].

In embodiments of these kits the CD1d-restricted NKT cell epitope motif has the sequence [FWY]-X(2)-[VILM]-X(2)-[FWY] [SEQ ID NO:28].

In embodiments of these kits said MHC class II T cell epitope is a promiscuous epitope binding to one or more HLA-DR1 molecules.

In embodiments of these kits the MHC class II T cell epitope has the sequence X1X2MATX6LLM [SEQ ID NO:29], wherein X1 and X2 are independently selected from V, I, L, M, Y, H, F and W, and X6 is R or P.

An aspect of the invention relates to the above kits for use as a medicament.

An aspect of the invention relates to polypeptides comprising:
b1) a therapeutic protein, and
b2) an MHC class II T cell epitope or a CD1d-restricted NKT cell peptide epitope, wherein the epitope sequence is a sequence which differs from the sequence of the protein of b1.
for use as a medicament in an individual who has been previously treated with a peptide comprising:
a1) said MHC class II T cell epitope or said CD1d-restricted NKT cell epitope and
a2) immediately adjacent to said epitope or separated by at most 7 amino acids from said epitope a sequence with a [CST]-X(2)-C [SEQ ID NO:7] or C-X(2)-[CST] [SEQ ID NO:8] oxidoreductase motif sequence.

An aspect of the invention relates to expression vectors encoding a polypeptide comprising:
b1) a therapeutic protein, and
b2) an MHC class II T cell epitope or a CD1d-restricted NKT cell peptide epitope, wherein the epitope sequence is a sequence which differs from the sequence of the protein of b1.
for use as a medicament in an individual who has been previously treated with a peptide comprising:
a1) said MHC class II T cell epitope or said CD1d-restricted NKT cell epitope and
a2) immediately adjacent to said epitope or separated by at most 7 amino acids from said epitope a sequence with a [CST]-X(2)-C [SEQ ID NO:7] or C-X(2)-[CST] [SEQ ID NO:8] oxidoreductase motif sequence.

An aspect of the invention relates to viral vectors for gene therapy or gene vaccination comprising in the backbone a polynucleotide sequence encoding a protein comprising:
b1) a viral protein for the function and maintenance of the vector, and
b2) an MHC class II T cell epitope or a CD1d-restricted NKT cell epitope, wherein the epitope sequence is a sequence which differs from the sequence of the protein of b1,
for use as a medicament in an individual who has been previously treated with a peptide comprising:
a1) said MHC class II T cell epitope or said CD1d-restricted NKT cell epitope and
a2) immediately adjacent to said epitope or separated by at most 7 amino acids from said epitope a sequence with a [CST]-X(2)-C [SEQ ID NO:7] or C-X(2)-[CST] [SEQ ID NO:8] oxidoreductase motif sequence

An aspect of the invention relates to a peptide comprising:
a1) an MHC class II T cell epitope or a CD1d-restricted NKT cell peptide epitope, and
a2) immediately adjacent to said epitope or separated from said epitope by at most 7 amino acids a sequence with a [CST]-X(2)-C [SEQ ID NO:7] or C-X(2)-[CST] [SEQ ID NO:8] oxidoreductase motif sequence, for preventing an immune response against a therapeutic protein or against a viral vector protein with said MHC class II T cell epitope or said CD1d binding peptide epitope,
wherein said epitope has a sequence which does not occur in the sequence of the therapeutic protein or of the viral vector protein.

An aspect of the invention relates to methods for preparing kits of parts of polypeptides, comprising
a) preparing a modified therapeutic protein by introducing into the sequence of said protein the sequence of an MHC class II T cell epitope or a CD1d-restricted NKT cell epitope, which epitope sequence is a sequence which does not occur in the unmodified protein,
b) preparing a peptide comprising:
   the MHC class II T cell epitope or a CD1d-restricted NKT cell peptide epitope of a), and
   immediately adjacent to said epitope or separated by at most 7 amino acids from said epitope a [CST]-X(2)-C [SEQ ID NO:7] or C-X(2)-[CST][SEQ ID NO:8] oxidoreductase motif sequence.

An aspect of the invention relates to methods for preparing kits of parts, comprising
a) preparing a vector comprising a polynucleotide sequence encoding a modified therapeutic protein or a modified viral vector protein for the function and maintenance of the vector by introducing into the sequence of said proteins the sequence of an MHC class II T cell epitope or a CD1d-restricted NKT cell peptide epitope, which epitope sequence is a sequence which does not occur in the unmodified protein,
b) preparing a peptide comprising:
   the MHC class II T cell epitope or a CD1d-restricted NKT cell peptide epitope of a), and
   immediately adjacent to said epitope or separated by at most 7 amino acids from said epitope a [CST]-X(2)-C [SEQ ID NO:7] or C-X(2)-[CST][SEQ ID NO:8] oxidoreductase motif sequence.

An aspect of the invention relates to peptides with a length of between 12 and 100 amino acids comprising:
a modified CLIP sequence with motif X1X2MATX6LLM [SEQ ID NO:29], wherein X1 and X2 are independently selected from V, I, L, M, F, H, Y and W, and wherein X6 is R or P, and
immediately adjacent to said modified CLIP sequence or separated by at most 7 amino acids from said modified CLIP sequence a [CST]-X(2)-C [SEQ ID NO:7] or C-X(2)-[CST] [SEQ ID NO:8] oxidoreductase motif sequence.

In embodiments hereof the CLIP sequence is selected from the group consisting of FFMATRLLM [SEQ ID NO:30], WWMATRLLM [SEQ ID NO:31], WFMATRLLM [SEQ ID NO:32], FWMATRLLM [SEQ ID NO:33], FFMATPLLM [SEQ ID NO:34], WWMATPLLM [SEQ ID NO:35], WFMATPLLM [SEQ ID NO:36] and FWMATPLLM [SEQ ID NO:37].

An aspect of the invention relates to therapeutic proteins or polynucleotides encoding a therapeutic protein or encoding a viral vector protein, characterised in the presence of an MHC class II T cell epitope, which epitope has a sequence which does not occur in the sequence of the therapeutic protein or of the viral vector protein wherein said MHC class II T cell epitope has a sequence with motif X1X2MATX6LLM [SEQ ID NO:29], wherein X1 and X2 are independently selected from V, I, L, M, F, H, Y and W, and wherein X6 is R or P.

An aspect of the invention relates to expression vectors comprising a multiple cloning site for the in frame insertion of a polynucleotide encoding a therapeutic protein, characterised in the presence of a nucleotide sequence encoding a promiscuous MHC class II T cell epitope or a CD1d-restricted NKT cell epitope, such that upon insertion of said polynucleotide of said therapeutic protein in said expression vector, a fusion protein is encoded and expressed comprising said therapeutic protein fused to said promiscuous MHC class II T cell epitope or said CD1d-restricted NKT cell epitope.

In embodiments hereof the expression vector is a mammalian expression vector.

In embodiments the expression vector comprises a sequence encoding a therapeutic protein in frame with a sequence encoding a promiscuous MHC class II T cell epitope or a CD1d-restricted NKT cell epitope, under the control of transcription and translation elements, allowing the expression of a fusion protein comprising said therapeutic protein fused to said promiscuous MHC class II T cell epitope or said CD1d-restricted NKT cell epitope, wherein said epitope has a sequence which differs from the sequence of the therapeutic protein.

In certain embodiments the MHC class II T cell epitope has a sequence with motif X1X2MATX6LLM [SEQ ID NO:29], wherein X1 and X2 are independently selected from V, I, L, M, F, H, Y and W, and wherein X6 is R or P.

In embodiments hereof the sequence is selected from the group consisting of FFMATRLLM [SEQ ID NO:30], WWMATRLLM [SEQ ID NO:31], WFMATRLLM [SEQ ID NO:32], FWMATRLLM [SEQ ID NO:33], FFMATPLLM [SEQ ID NO:34], WWMATPLLM [SEQ ID NO:35], WFMATPLLM [SEQ ID NO:36] and FWMATPLLM [SEQ ID NO:37].

The invention relates to combinations of polypeptides of:
a) a modified therapeutic protein or a modified viral vector protein characterised in that the modification is the presence of an MHC class II T cell epitope or an CD1d-restricted NKT cell peptide epitope, which epitope has a sequence which does not occur in the unmodified sequence of the therapeutic protein or of the viral vector protein, and
b) a peptide comprising the MHC class II T cell epitope or the CD1d-restricted NKT cell peptide epitope defined in a) and immediately adjacent to the epitope or separated by at most 7 amino acids from the epitope a sequence with a [CST]-X(2)-C [SEQ ID NO:7] or C-X(2)-[CST] [SEQ ID NO:8] oxidoreductase motif sequence.

In embodiments of the combinations, the protein in a) is a fusion protein of the therapeutic protein or of the viral vector protein fused to an MHC class II T cell epitope or an CD1d-restricted NKT cell peptide epitope.

In embodiments of the combinations, the protein in a) is modified with at least 2 different epitopes, and in b) at least 2 peptides are present, each peptide comprising an epitope as defined in a).

In embodiments of the combinations, the oxidoreductase motif sequence is C-X(2)-C [SEQ ID NO:2].

In embodiments of the combinations, in b) the epitope and the redox motif are separated by at most 4 amino acids.

Embodiments of the CD1d-restricted NKT cell peptide epitope motif are [FWYHT]-X(2)-[VILM]-X(2)-[FWYHT] [SEQ ID NO:1], or [FWYH]-X(2)-[VILM]-X(2)-[FWYH] [SEQ ID NO:27] and [FWY]-X(2)-[VILM]-X(2)-[FWY][SEQ ID NO:28].

In a typical embodiment, the T cell epitope is a promiscuous epitope binding to one or more HLA-DR1 molecules, which preferably binds to at least HLA-DR1*0101, HLA-DR1*0102, and HLA-DR1*0302.

In specific embodiments, the T cell epitope has the sequence X₁X₂MATX₆LLM [SEQ ID NO:29], wherein X₁ and X₂ are independently selected from V, I, L, M, Y, H, F and W, and X₆ is R or P. Examples hereof are FFMATRLLM [SEQ ID NO:30], WWMATRLLM[SEQ ID NO:31], WFMATRLLM [SEQ ID NO:32], or FWMATRLLM [SEQ ID NO:33].

The invention relates to the combination of polypeptides described in the above aspect for use as a medicament.

The invention relates to modified therapeutic protein or a modified viral vector protein wherein the modification is the presence of an MHC class II T cell epitope or a CD1d-restricted NKT cell peptide epitope, which epitope has a sequence which does not occur in the unmodified sequence of the therapeutic protein or of the viral vector protein, for use as a medicament in an individual who has been previously treated with a peptide comprising the MHC class II T cell epitope or the CD1d-restricted NKT cell peptide epitope and immediately adjacent to the epitope or separated by at most 7 amino acids from the epitope a sequence with a [CST]-X(2)-C [SEQ ID NO:7] or C-X(2)-[CST] [SEQ ID NO:8] oxidoreductase motif sequence.

The invention relates to a peptide comprising an MHC class II T cell epitope or a CD1d binding peptide epitope and comprising immediately adjacent to the epitope or separated by at most 7 amino acids from the epitope a sequence with a [CST]-X(2)-C [SEQ ID NO:7] or C-X(2)-[CST] [SEQ ID NO:8] oxidoreductase motif sequence, for preventing an immune response against a therapeutic protein or against a viral vector protein with the MHC class II T cell epitope or the CD1d binding peptide epitope,
wherein the epitope has a sequence which does not occur in the wild type sequence of therapeutic protein or the viral vector protein, and optionally
wherein the protein does not contain a sequence with a [CST]-X(2)-C [SEQ ID NO:7] or C-X(2)-[CST] [SEQ ID NO:8] in the 7 amino sequence at the N terminal or C terminal part of the epitope sequence.

The invention relates to methods for preparing a modified therapeutic protein or a modified viral vector protein comprising the step of introducing into the sequence of the protein the sequence of an MHC class II T cell epitope or a CD1d-restricted NKT cell peptide epitope, which is a sequence which does not occur in the unmodified protein.

Typically, the epitope sequence is attached to the protein to obtain a fusion protein.

The invention relates to peptides with a length of between 12 and 100 amino acids comprising a modified CLIP sequence with motif X₁X₂MATX₆LLM [SEQ ID NO:29], wherein X₁ and X₂ are independently selected from V, I, L, M, F, H, Y and W, and wherein X₆ is R or P. Examples hereof are FFMATRLLM [SEQ ID NO:30], WWMATRLLM [SEQ ID NO:31], WFMATRLLM [SEQ ID NO:32], FWMATRLLM [SEQ ID NO:33], FFMATPLLM [SEQ ID NO:34], WWMATPLLM [SEQ ID NO:35], WFMATPLLM [SEQ ID NO:36] and FWMATPLLM [SEQ ID NO:37].

In specific embodiments, these peptides further comprise a sequence with a [CST]-X(2)-C [SEQ ID NO:7] or C-X(2)-[CST] [SEQ ID NO:8] redox motif sequence, wherein the redox motif sequence and the modified clip sequence are separated by 0 to 7 amino acids, or 0 to 4 amino acids, or 0 to 2 amino acids.

A specific embodiment of the motif is C-X(2)-C [SEQ ID NO:2].

The invention relates to modified therapeutic proteins or a modified viral vector proteins characterised in that the modification is the presence of an MHC class II T cell epitope or an CD1d-restricted NKT cell peptide epitope, which epitope has a sequence which does not occur in the unmodified sequence of the therapeutic protein or of the viral vector protein.

### DETAILED DESCRIPTION

### Definitions

The distinction between the term “peptide” and “protein” is arbitrarily since both refer to polypeptides connected by peptide bonds, but which can comprise non-amino acid structures (like for example a linking organic compound). Polypeptides can contain any of the conventional 20 amino acids or modified versions thereof as obtained with posttranslational modifications, or can contain non-naturally occurring amino-acids incorporated by chemical peptide synthesis or by chemical or enzymatic modification (e.g. physiological amino acids). As used herein peptide will be used to refer to a molecule comprising an amino acid sequence of between 2 up to 20, 30, 50, 75 or 100 amino acids.

The term “antigen” as used herein refers to a structure of a macromolecule, typically a protein (with or without polysaccharides) or a proteic composition comprising one or more hapten (s), and comprising T cell epitopes. The term “antigenic protein” as used herein refers to a protein comprising one or more T cell epitopes. An auto-antigen or auto-antigenic protein as used herein refers to a human or animal protein present in the body, which elicits an immune response within the same human or animal body.

The term “food or pharmaceutical antigenic protein” refers to an antigenic protein in a food or pharmaceutical product, such as in a vaccine.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B or T cell lymphocyte, and which is able, by this binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. Whether an epitope is dominant, sub-dominant or minor depends on the immune reaction elicited against the epitope. Dominance depends on the frequency at which such epitopes are recognised by T cells and are able to activate them, among all the possible T cell epitopes of a protein. A T cell epitope is an epitope recognised by MHC class II molecules, which consists of a sequence of typically 9 amino acids which fit in the groove of the MHC II molecule (the length of the peptide fitting in the groove of the MHC II molecule may be 8 or 10 amino acids for some peptide/MHCII complexes). Within a peptide sequence representing a 9 amino acid T cell epitope, the amino acids in the epitope are numbered P1 to P9, amino acids N-terminal of the epitope are numbered P-1, P-2 and so on, amino acids C terminal of the epitope are numbered P+1, P+2 and so on.

“Motifs” of amino acid sequences are written herein according to the format of Prosite (Sigrist et al. (2002) Brief Bioinform. 3, 265-274). The symbol X is used for a position where any amino acid is accepted. Alternatives are indicated by listing the acceptable amino acids for a given position, between square brackets (‘[ ]’). For example: [CST] stands for an amino acid selected from Cys, Ser or Thr. Amino acids which are excluded as alternatives are indicated by listing them between curly brackets (‘{ }’). For example: {AM} stands for any amino acid except Ala and Met. The different elements in a motif are separated from each other by a hyphen -. Repetition of an identical element within a motif can be indicated by placing behind that element a numerical value or a numerical range between parentheses. For example: X(2) corresponds to X-X, X(2,3) corresponds to X-X or X-X-X, A(3) corresponds to A-A-A.

The term “CD1d-restricted NKT cell peptide epitope” or “CD1d-restricted NKT cell peptide epitope” refers to a part of an antigenic protein that is specifically bound by a CD1d molecule, expressed at cell surface and recognized by a NKT cell. The word “peptide” in this definition may be used to emphasise the difference with prior art binding CD1d binding compounds such as ceramides.

The CD1d-restricted NKT cell peptide epitope has a general motif [FWYHT]-X(2)-[VILM]-X(2)-[FWYHT] [SEQ ID NO:1]. Alternative versions of this general motif have at position 1 and/or position 7 the alternatives [FWYH].

Alternative versions of this general motif have at position 1 and/or position 7 the alternatives [FWYT].

Alternative versions of this general motif have at position 1 and/or position 7 the alternatives [FWY].

Regardless of the amino acids at position 1 and/or 7, alternative versions of the general motif have at position 4 the alternatives [ILM]. The term “homologue” as used herein with reference to epitopes used in the context of the invention, refer to molecules having at least 50%, at least 70%, at least 80%, at least 90%, at least 95% or at least 98% amino acid sequence identity with a naturally occurring epitope, thereby maintaining the ability of the epitope to bind an antibody or cell surface receptor of a B and/or T cell. Specific homologues of an epitope correspond to a natural epitope modified in at most three, more particularly in at most 2, most particularly in one amino acid.

The term “derivative” as used herein with reference to peptides of the invention refers to molecules which contain at least the peptide active portion (i.e. capable of detecting CD4+ T cell) and, in addition thereto comprises an additional part which can have different purposes such as stabilising a peptide or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. The sequence identity can be more than 70%, more than 80% more than 90% more than 95% more than 98%, or more than 99%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. For example, the nucleic acid encoding a peptide fragment thereof is a sequence encoding the peptide or fragment thereof originating from a mammal or corresponding to a mammalian, most particularly a human peptide fragment.

The term “organic compound having a reducing activity” refers in the context of this invention to compounds, more in particular amino acid sequences, with a reducing activity for disulfide bonds on proteins.

The reducing activity of an organic compound can be assayed for its ability to reduce a sulfhydryl group such as in the insulin solubility assay wherein the solubility of insulin is altered upon reduction, or with a fluorescence-labelled insulin. The reducing organic compound may be coupled at the amino-terminus side of the T-cell epitope or at the carboxy-terminus of the T-cell epitope. Generally the organic compound with reducing activity is a peptide sequence. Peptide fragments with reducing activity are encountered in thioreductases which are small disulfide reducing enzymes including glutaredoxins, nucleoredoxins, thioredoxins and other thiol/disulfide oxydoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They exert a reducing activity for disulfide bonds on proteins (such as enzymes) through redox active cysteines within conserved active domain consensus sequences: C-X(2)-C [SEQ ID NO:2], C-X(2)-S [SEQ ID NO:3], C-X(2)-T [SEQ ID NO:4], S-X(2)-C [SEQ ID NO:5], T-X(2)-C [SEQ ID NO:6](Fomenko et al. (2003) Biochem. 42, 11214-11225; Fomenko et al. (2002) Prot. Science 11, 2285-2296), in which X stands for any amino acid. Such domains are also found in larger proteins such as protein disulfide isomerase (PDI) and phosphoinositide-specific phospholipase C.

The term “natural” “wild type”, “native” when referring to a peptide or a sequence herein relates to the fact that the sequence is identical to a naturally occurring sequence or a fragment thereof. In contrast therewith the term “artificial” refers to a sequence or peptide which as such does not occur in nature and differs from the above natural/wild type/native sequence. Optionally, an artificial sequence is obtained from a natural sequence by limited modifications such as changing one or more amino acids within the naturally occurring sequence or by adding amino acids N- or C-terminally of a naturally occurring sequence. Amino acids are referred to herein with their full name, their three-letter abbreviation or their one letter abbreviation. An artificial sequence can also be obtained by chemically modifying amino acid side chains or by including non-natural amino acids.

The term “major histocompatibility antigen” refers to molecules belonging to the HLA system in man (H2 in the mouse), which are divided in two general classes. MHC class I molecules are made of a single polymorphic chain containing 3 domains (alpha 1, 2 and 3), which associates with beta 2 microglobulin at cell surface. Class I molecules are encoded by 3 loci, called A, B and C in humans. Such molecules present peptides to T lymphocytes of the CD8+ subset. Class II molecules are made of 2 polymorphic chains, each containing 2 chains (alpha 1 and 2, and beta 1 and 2). These class II molecules are encoded by 3 loci, DP, DQ and DR in man. Hereof the HLA-DR molecule is the most prevalent in humans. The frequency of alleles in different nationalities and ethnic can be obtained from allelefrequencies.net (Gonzalez-Galarza et al. (2015) Nucl. Acid Res. 28, D784-D788.

“Gene therapy” can be defined as the insertion, ex vivo or in vivo, of a gene or genes into individual cells or groups of cells (such as tissues or organs) with the purpose to provide a missing gene or allele or to replace a mutant gene or a mutant allele with a functional copy delivered by the gene therapy. The “therapeutic gene” is delivered via a carrier called a vector.

The most common vector is a viral vector. Upon infection of targeted cells with the viral vector carrying the therapeutic gene, the viral vector unloads its genetic material including the therapeutic gene into the target cells, followed by the generation of the functional protein(s) encoded by the therapeutic gene. Cells targeted by gene therapy can be either somatic cells or germ cells or cell lines. In addition, gene therapy refers to the use of vectors to deliver, either ex vivo or in vivo, a gene that requires overexpression or ectopic expression in a cell or group of cells. The vector can facilitate integration of the new gene in the nucleus or can lead to episomal expression of that gene.

“Gene vaccination” can be defined as the administration of a functional gene (i.e., capable of expressing the protein encoded by the gene) to a subject for the purpose of vaccinating a subject. Thus, gene vaccination (or DNA vaccination) is a variant of the more classical vaccination with peptides, proteins, attenuated or killed germs, etc. Gene vaccination can be performed with naked DNA or, of particular interest in the context of the present invention, with viral vectors.

The term “viral vector protein” when used herein refers to any protein or peptide derived from the backbone of a viral vector as such and which are required for the function and maintenance of the vector. It does not refer to the therapeutic gene which is cloned into the vector. Typically such viral vector proteins are antigenic and comprise one or more epitopes such as T-cell epitopes. A well-known example is the capsid protein. Several viruses are currently used for gene therapy, both experimental and in man, including RNA viruses (gamma-retroviruses and lentiviruses) and DNA viruses (adenoviruses, adeno-associated viruses, herpes viruses and poxviruses).

The term “allofactor” or “alloantigen” refers to a protein, peptide or factor (i.e., any molecule) displaying polymorphism when compared between 2 individuals of the same species, and, more in general, any protein, peptide or factor that is inducing an (alloreactive) immune response in the subject receiving the allofactor.

The term “alloreactivity” refers to an immune response that is directed towards allelic differences between the graft recipient and the donor. Alloreactivity applies to antibodies and to T cells. The present invention relies entirely on T cell alloreactivity, which is based on T cell recognition of alloantigens presented in the context of MHC determinants as peptide-MHC complexes.

The term “promiscuous” refers to epitopes which carry the property of being able to bind to different MHC class II molecules in order to cover a substantial percentage of the envisaged population. A substantial percentage is at least 50%, or least 60%, at 75%, at least 90% or even at least 95% of the envisaged population.

The envisaged population can be defined as individuals of one or more countries or of one or more regions or continents, or as members of an ethnic group.

Alternatively, a promiscuous epitope can be defined as binding to at least 7, 8, 9 or 10 of the 15 most prevalent HLA DR alleles in the envisaged population.

Promiscuous epitopes can be natural sequences as occurring in an antigen or modified by substitution of one or more amino acids (2, 3 or 4) of a natural sequence occurring in an antigen, or completely artificial, including non-physiological amino acids, amino acids containing modified side chains, or small compounds.

The term “universal epitope” also occurs in the art. However this relates to an epitope sequence which is encountered in different antigens. Such an universal epitope may or may not bind to different MHC molecules and alleles.

One aspect of the present invention relates to modified version of therapeutic proteins or of viral vector proteins with an added MHC class II T cell epitope or with an added CD1d-restricted NKT cell peptide epitope. This is typically done by generating fusion proteins of the antigen and the epitope sequence, although the epitope sequence can be introduced as well in the protein itself.

Herein the 9 amino acid T cell epitope sequence or the 7 amino acid CD1d-restricted NKT cell peptide epitope sequence is not a fragment of the wild type therapeutic protein or of the viral vector protein.

This can be achieved by a modification of an epitope sequence as occurring in the therapeutic protein/viral vector protein, or by the use of an epitope sequence as occurring in another antigen (human or non-human) or by the use of a designed sequence with low or no sequence identity to any existing antigen epitope sequence.

The MHC class II T cell epitope can be a promiscuous epitope. The rationale for this choice is explained later on in more detail.

The modified therapeutic protein/viral vector protein could itself elicit the generation of CD4+ cytotoxic cells, if in this protein a [CST]-X(2)-C [SEQ ID NO:7] C-X(2)-[CST] [SEQ ID NO:8] motif sequence is within 4, typically within 7 amino acids from the sequence of the introduced class II T cell epitope or the introduced CD1d-restricted NKT cell peptide epitope. This can happen when the modification includes, apart from the introduction of the epitope sequence, also the introduction of a oxidoreductase sequence. In some proteins which contain in their wild type sequence an oxidoreductase sequence, the protein can be engineered to contain the epitope sequence in the proximity (separated by at most 7 or at most 4 amino acids) of the naturally occurring oxidoreductase sequence.

If such activity is not required by the modified therapeutic protein/viral vector protein, the modification is limited to the introduction of the epitope sequence. If the modified therapeutic protein/viral vector protein contains in its wild type sequence an oxidoreductase sequence, the added epitope sequence is introduced in the modified protein such that the added epitope and the existing oxidoreductase motif are separated by at least 4, or at least 7 amino acids.

The epitope can occur in a fusion protein N terminal of the antigen, or C terminal of the antigen, depending on the impact of the added epitope sequence on the function of the protein. In specific embodiments two or more different epitopes are added to the antigen.

If appropriate a linker sequence can be inserted between the epitope sequence and the sequence of the native antigen.

It is also envisaged to generate modified versions of an antigen wherein the epitope sequence is fused internally in the sequence at a region which is not critical for the function of the protein. In alternative embodiments a foreign epitope sequence is generated by mutating and/or adding one or more amino within the sequence of the antigen.

The length of the protein with the introduced epitope sequence (added as a fusion protein, or internal) is mainly defined by the antigen and is not a limiting feature. The antigen can be very small in case of peptide hormones. The impact of mutations within a protein is likely larger in shorter proteins, and encourages to consider fusion proteins for proteins with a length of less than 100 or less than 250 amino acids.

The therapeutic agent of the present invention includes any peptide or protein agent used to compensate for the absence of a physiological agent or to alter, modify, stop or slow down a disease process.

Such therapeutic agents include:
1. replacement agents for coagulation defects, including factor VIII, factor IX and factor X deficiencies, administration of factor VII to correct a deficiency or as a therapeutic agent in trauma, surgery, heart failure or in the treatment of patients affected by haemophilia and producing inhibitory antibodies which preclude administration of the missing factor (factor VIII or factor IX)
2. fibrinolytic agents, including staphylokinase or tissue plasminogen activator (tPA) administered in stroke and cardiac infarction
3. hormones such as growth hormone or insulin to treat nanism and insulin-dependent diabetes mellitus
4. cytokines and growth factors, such as interferon-alpha, interferon-gamma, GM-CSF and G-CSF; cytokine receptors such as IL-6 receptor and IL-1b receptor in the treatment of rheumatoid arthritis
5. antibodies for the modulation of immune responses, including anti-IgE antibodies in allergic diseases, anti-CD3 and anti-CD4 antibodies in graft rejection and a variety of autoimmune diseases, anti-CD20 antibodies in non-Hodgkin lymphomas anti-cytokine or cytokine receptor (e.g. anti-IL6R). Humanized therapeutic antibodies are human except for the epitope binding site (CDRs), usually derived from a mouse sequence. The present invention aims to eliminate the capacity to mount a response towards the non-human part of the antibody.
6. erythropoietin in renal insufficiency.
7. replacement therapy with enzymes, including alpha-galactosidase A (Fabry disease), beta-glucocerebrase (type 1 Gaucher disease) and alpha-glucosidase (Pompe disease).

Further to the above section on proteins with an added MHC class II T cell epitope or CD1d-restricted NKT cell peptide epitope, another aspect of the present invention relates to a combination of a protein as described above, and another peptide comprising a 4 amino acid oxidoreductase sequence and comprising the same epitope sequence that has been added to the therapeutic protein or the viral vector.

In this peptide, the oxidoreductase sequence has the motif [CST]-X(2)-C [SEQ ID NO:7] or C-X(2)-[CST] [SEQ ID NO:8]. This motif encompasses the alternatives C-X(2)-C [SEQ ID NO:2], S-X(2)-C [SEQ ID NO:5], T-X(2)-C [SEQ ID NO:6], C-X(2)-S [SEQ ID NO:3] and C-X(2)-T [SEQ ID NO:4]. A particular choice of the motif is C-X(2)-C [SEQ ID NO:2].

In the motif of reducing compounds, C represents either cysteine or another amino acids with a thiol group such as mercaptovaline, homocysteine or other natural or non-natural amino acids with a thiol function. In order to have reducing activity, the cysteines present in the motif should not occur as part of a cystine disulfide bridge.

The amino acid X in the redox can be any natural amino acid, or can be a non-natural amino acid. X can be an amino acid with a small side chain such as Gly, Ala, Ser or Thr. In particular versions at least one X in the redox motif is His, Pro or Tyr. In particular versions X is not Cys, in other particular versions X is not W, F or Y.

In particular conditions, peptides are provided comprising one epitope sequence and a motif sequence. The motif can occur once or several times (2, 3, 4 or even more times) in the peptide, for example as repeats of the motif which can be spaced from each other by one or more amino acids, as repeats which are adjacent to each other, or as repeats which overlap with each other.

The epitope sequence and the 4 amino acid oxidoreductase sequence do not overlap and are separated by a linker sequence of 7, 6, 5, 4, 3, 2 or 1 amino acids, or are immediately adjacent to each other (thus no linker sequence or linker of 0 amino acids). Typical ranges are a linker of 0 to 2 amino acids, a linker of 0 to 4 amino acids, or a linker of 0-7 amino acids.

When the epitope sequence in the peptide is a fragment of an antigen, the amino acids in the linker are typically the amino acid(s) which flank the epitope in the antigen. Alternatively the amino acids in a linker are Gly and/or Ser.

The redox motif can be as well at the N terminus or at the C terminus of the epitope sequence.

The peptide with redox motif sequence and epitope sequence can be as short as 12 or 13 amino acids for peptides with an MHC class II T cell epitope or 11 amino acids for peptides with a CD1d-restricted NKT cell peptide epitope, up to 20, 30, 40, 50, 75 or 100 amino acids, depending on the number of amino acids between epitope and oxidoreductase motif, and the number of flanking amino acids at N and/or C terminus of the “epitope+linker+redox motif” sequence or “redox motif+linker+epitope” sequence.

Thus depending on the embodiment of choice the epitope is a MHC class II epitope or a CD1d-restricted NKT cell peptide epitope. CD1d-restricted NKT cell peptide epitopes can be used to generate cytolytic CD4+ T cells via NKT cells.

CD1d-restricted NKT cell peptide epitopes have the advantage that they will in all individuals bind to the CD1d binding since there is no polymorphism for this protein.

The use of proteins with an added MHC class II T cell epitope or CD1d-restricted NKT cell peptide epitope in combination with a peptide comprising this epitope and a redox motif allows to uncouple the epitope sequence used to elicit the cytolytic CD4+ cell population from epitope sequences within the antigen itself.

This is especially the case for fusion proteins of a protein fused to an epitope, since this requires no modification to the sequence of the protein itself. Modifying the therapeutic protein to contain the new epitope sequence is a task that has to be optimised for each protein.

Uncoupling the epitope sequence from the antigen sequence allows to design a tailor made epitope sequence. This sequence can be optimised to improve solubility and stability, decrease degradation, and the like. Apart from the choice of certain amino acids in the sequence, other alterations can be envisaged such as the incorporation of non-natural amino acids or D-amino acids during peptide synthesis, or posttranslational modifications such as citrullination, acetylation and sulfatation.

An epitope sequence can also be optimised to assure that the MHC class II T cell epitope will bind to different HLA proteins such that the will be recognised by CD4+ T cells in a large proportion of subjects. Such epitopes are known in the art as promiscuous epitopes.

Examples of such promiscuous epitopes can be found in viral agents such as measles or hepatitis C, mycobacteria, tumor-related viral antigens or even in autologous proteins. Algorithms to determine the binding of an epitope sequence with an HLA molecule or allele, and to identify promiscuous epitopes in a protein sequence are known. Candidate promiscuous epitopes can be tested on different MHC molecules.

As an alternative or in addition to the use of a promiscuous MHC class II T cell epitope, two or more peptides can be mixed for vaccination, each containing a thioreductase motif and MHC class II T cell epitope, so as to cover the largest possible numbers of MHC class II determinants. In this case the antigen will also carry the two or more epitope sequences used in the peptide mixture (typically in the form of a fusion protein).

In a specific embodiment a combination of an MHC Class II T cell epitope and a CD1d-restricted NKT cell peptide epitope is used.

In a specific embodiment, the promiscuous epitope is derived from the CLIP (CLass II associated Invariant chain Peptide (KM₁R₂MATP₆LLMQAL) [SEQ ID NO:9] sequence obtained by proteolytic cleavage of the invariant chain. CLIP is protecting the hydrophobic peptide-binding groove of MHC class II molecule until it is displaced by competition with a peptide of higher affinity. CLIP protects all nascent MHC class II molecules from the DR, DP or DQ family, and as such is the most illustrative example of a promiscuous epitope. However, the affinity of CLIP for class II molecules is weak, so that there is no presentation of MHC class II molecules with CLIP at the surface of an antigen-presenting cell. The DM protein catalyses the replacement of CLIP by alternative peptides for surface presentation (Pos et al. (2012) Cell 151, 1557-1568).

It is an aspect of the invention that replacement of the first CLIP amino acid residue located in the P1 pocket of the MHC class II molecule by a hydrophobic residue such as F, W, H or Y or V, I, L, or M is sufficient as to prevent the complete replacement by an alternative peptide and allow presentation of modified CLIP at the surface of an antigen-presenting cell. This results in activation of CD4+ T cells. In an alternative version, also the second amino acid residue of the MHC binding fragment of CLIP is replaced by a hydrophobic residue.

In addition, the proline residue located in position 6 can be mutated from P to R to further increase its binding affinity.

Accordingly the present invention provides peptides comprising a modified version of the MHC class II binding region of the Clip peptide represented by the general sequence motif [VILMFWYH]₁[RVILMFWYH]₂MAT[PR]₆LLM [SEQ ID NO:10].

In such modified Clip peptides, independent from each other P1, can also be [FWHY] [FWH] or [FW], P2 can be [RFWHY], RFW or R, P6 can be P. In specific embodiments, P1 and P6 are modified with one of the above possibilities and P2 Arginine is not modified.

Peptides can be made entirely artificially using sequences which fit closely in a majority of MHC class II molecules. One example of this is provided by the PADRE peptide (aKXVAAWTLKAAaZC (a=D-Alanine, X=I-cyclohexylalanine, Z=aminocaproic acid) [SEQ ID NO:11] (Alexander et al. (2000) J. Immunol. 64, 1625-1633). Such artificial promiscuous peptides can be made from computer algorithms taking into account the properties of the amino acid residues and those of the MHC class II molecules to obtain the best fit. One example of such algorithms is provided by ProPred (Sigh and Raghava (2001), Bioinformatics 17, 1236-1237). Other examples of algorithms are given below.

Promiscuous epitopes are also encountered in tetanus toxoid peptide (830-843) or influenza haemagglutinin, HA(307-319). Methods for the detection of promiscuous epitopes by in silico and cell based assays are described in Mustafa et al. (2014) PLoS One 9, e103679; Grzybowska-kowalczyk (2015) Thorax 69, 335-345; Grabowska et al. (2014) Int. J. Cancer 224, 1-13; Fraser et al. (2014). Vaccine 32, 2896-2903.

Non-natural (or modified) T-cell epitopes can further optionally be tested on their binding affinity to MHC class II molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that class II molecule. Peptides to be assessed for class II binding are then incubated at different concentrations and their capacity to displace the reference peptide from its class II binding is calculated by addition of neutravidin. Methods can be found in for instance Texier et al. (2000) J. Immunol. 164, 3177-3184.

Additionally and/or alternatively, one or more in silico algorithms can be used to identify a T cell epitope sequence within a protein. Suitable algorithms include, but are not limited to those found on the following websites:
cvc. dfci.harvard.edu/balbc/
syfpeithi.de/
abi.inf.uni-tuebingen.de/Services/SVMHC
bio. dfci.harvard.edu/Tools/antigenic.html;
ddg-pharmfac.net/mhcpred/MHCPred/
immunax. dfci.harvard.edu (PEPVAC)
epivax.com/epimatrix/

More particularly, such algorithms allow the prediction within an antigenic protein of one or more nonapeptide sequences which will fit into the groove of an MHC II molecule.

An example of determining the promiscuous nature of MHC class II peptides is described in WO2015/033140 using the method developed by Stumiolo et al. (1999) Nat. Biotechnol. 17, 555-561) available at www.iedb.org. Herein HLA Class II alleles considered for the analysis are HLA-DRA*01:01/HLA-DRB1*01:01, DRA*01:01/HLA-DRB1*03:01, DRA*01:01/HLA-DRB1*04:01, DRA*01:01/HLA-DRB1*07:01, DRA*01:01/HLA-DRB1*08:02, DRA*01:01/HLA-DRB1*11:01, DRA*01:01/HLA-DRB1*13:01 and DRA*01:01/HLA-DRBI*15:01 respectively considered as representative members of HLA-DR1, HLA-DR3, HLA-DR4, HLA-DR7, HLA-DR8, HLA-DR11, HLA-DR13 and HLA-DR15 antigen groups.

It is a further aspect of the present invention that class II-restricted epitopes, be them natural or artificial, can be used as a vaccine to elicit cytolytic CD4+ T cells. Subsequent administration of a therapeutic protein to which the same epitope, but without thioreductase motif, is added leads to activation of cytolytic CD4+ T cells obtained by vaccination. This results 10 in the prevention of an immune response to the therapeutic agent.

The methods of the present invention relate to combination therapies wherein a peptide with a redox motif and a T cell epitope or a CD1d-restricted NKT cell peptide epitope are used to generate a population of cytotoxic CD4+ T cells, or cytotoxic CD4+ NKT cells, respectively, which kill antigen-presenting cells presenting an antigen which contains the epitope sequence used in the peptide with redox motif.

In this way a subject is vaccinated and an immune response against a later administered antigenic protein with the epitope is prevented.

The use of promiscuous class II-restricted T cell epitopes and CD1d binding peptide epitopes for vaccination before administration of therapeutic agents are therefore an aspect of the present invention. The use of therapeutic agents containing the same epitope as that used for vaccination but without thioredox motif are a part of this aspect of the invention.

Peptides and therapeutic agents are used to treat subjects in need of the therapeutic agent. Thus, in one application of the invention the subject is immunized with a peptide encompassing a promiscuous epitope and a thioreductase motif. Typically, such immunization is carried out by subcutaneous administration of a peptide adsorbed or dissolved in an adjuvant. The immunized subject is then treated with the therapeutic agent for which he/she is in need, the agent containing the same promiscuous epitope as the one included in the peptide used for vaccination, but without the thioreductase motif. The immune response towards the therapeutic agent is prevented due to the vaccination procedure through which cytolytic CD4+ T cells specific for the promiscuous epitope have been elicited.

Administration of the peptide containing a thioredox motif can be carried out by direct immunization. Alternatively, administration may consist in cells obtained from the subject in need for a therapeutic agent, exposure of cells to the peptide in vitro for eliciting and expanding cytolytic CD4+ T cells and re-administration to a subject.

T cell epitopes of the present invention are thought to exert their properties by increasing the strength of synapse formation by creating a disulfide bridge between the thioreductase motif and the CD4 molecule. This mechanism of action is substantiated by experimental data (see examples below), but there is no intention to restrict the present invention to this specific mechanism of action.

It should be obvious for those skilled in the art that multiple variations of this sequence of events can be delineated, depending on the type and frequency at which the therapeutic agent has to be administered and the clinical condition of the subject in need for a therapeutic agent.

In one of these variations it might be more appropriate to treat the subject first by an infusion of his/her own cells after exposure of such cells to the epitope containing the thioreductase motif in an in vitro cell culture. This could be a preferred method in subjects under immunosuppressive treatment, which would prevent the development of an immune response to the peptide administered with an adjuvant.

Peptide administration can be envisaged by any route, with a preferred route being subcutaneous.

Peptides can be made by chemical synthesis, which allows the incorporation of non-natural amino acids and/or of chemically-modified amino acids. Examples of chemically-modified amino acids are encountered in pathological conditions, e.g. glycosylation and citrullination of epitopes in rheumatoid arthritis, deamidation in celiac disease and formation of an intra-epitope disulfide bridge in insulin-dependent diabetes mellitus. However, there are numerous possibilities of modifying amino acid side chains and the above examples are not meant to be exhaustive.

Polypeptides can be generated using recombinant DNA techniques, in bacteria, yeast, insect cells, plant cells or mammalian cells. Peptides of a shorter length can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine.

Chemical peptide synthesis methods are well described and peptides can be ordered from companies such as Applied Biosystems and other companies. Peptide synthesis can be performed as either solid phase peptide synthesis (SPPS) or contrary to solution phase peptide synthesis. The best-known SPPS methods are t-Boc and Fmoc solid phase chemistry. During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. In certain situations, such protecting groups can be left on the peptide after synthesis.

Alternatively, peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

In embodiments of the present invention, expression vectors are provided wherein
an MHC class II epitope or CD1d-restricted NKT cell peptide is cloned into the vector, typically upstream or downstream of the multiple cloning of a commercially available expression vector. The in frame insertion of the DNA encoding for a therapeutic protein allows the expression of an epitope tagged protein, comparable to e.g. an His tagged protein or HA tagged protein. Such modified expression vectors can be generated using standard molecular biology techniques.

The attachment of the epitope tag results in a fusion protein of the epitope with a therapeutic protein of choice for replacement therapy. A single vector can be used for any therapeutic protein.

In order to administer the protein to a patient, the protein can be expressed using a bacterial, yeast, plant or mammalian vector. The choice for the type of expression system depends from protein to protein is known for most therapeutic proteins. The isolated protein is accordingly injection.

Alternative, DNA encoding the therapeutic protein fused to the epitope is cloned into a mammalian expression vector suitable for gene therapy in humans.

The physical and chemical properties of a peptide of interest (e.g. solubility, stability) are examined to determine whether the peptide is/would be suitable for use for applications as defined for the present invention. Typically this is optimised by adjusting the sequence of the peptide. Optionally, the peptide can be modified after synthesis (chemical modifications e.g. adding/deleting functional groups) using techniques known in the art. Optionally, peptides can be modified by posttranslational alteration. Examples of this are acetylation, sulfation, citrullination or phosphorylation of single of multiple amino acid residues.

The invention is now illustrated by the following examples, with no intention to restrict the invention to these examples.

### Example 1: Therapeutic Antibody

Many viruses contain universal class II restricted T cell epitopes, one example being the hepatitis C virus. The peptide sequences 1247 to 1261 (QGYK VLVLNPSVAA T) [SEQ ID NO:12] and 1535 to 1550 (TTVRLRA YMNTPGLPV) [SEQ ID NO:13], cover together more 12 of the 15 DRB haplotypes, representative of more than 85% of the general population.

A vaccination strategy making use of a mixture of two peptides encompassing the minimal binding sequence of class II-restricted epitopes and a thioreductase motif is therefore established.

Peptide 1247-1261 contains an MHC class II binding sequence at position 1251-1260 (underlined in SEQ ID NO:12].

Peptide 1535-1550 contains a minimal MHC class II binding sequence at positions 1542-1550 (underlined in SEQ ID NO:13].

Two peptides are prepared for vaccination wherein redox motif and epitope sequence are separated by a VR dipeptide linker:

[SEQ ID NO: 14]
CPYC-VR-VLVLNPSVAA,
and
[SEQ ID NO: 15]
CPYC-VR-YMNTPGLPV

Administration of a mixture of these two peptides adsorbed on aluminum hydroxide elicits specific CD4+ T cells with cytolytic properties.

Antibodies to CD20 are a recognized treatment for non-Hodgkin lymphoma. However, in a significant percentage of patients, this administration elicits specific antibodies which either preclude further administration or minimize efficacy.

The present invention provides a vaccination strategy to prevent such unwanted immunization.

The above 2 sequences from the hepatitis C virus, namely SEQ ID NO:12 and SEQ ID NO:13, are produced in line with the anti-CD20 antibody and positioned at the amino-terminal end of the heavy chain [SEQ ID NO:16]. Upon administration of this anti-CD20 antibody, cytolytic CD4+ T cells elicited previously by vaccination become activated and eliminate by apoptosis the presentation of determinants from the therapeutic antibody, thereby preventing immunization towards anti-CD20.

### Example 2: Erythropoietin

Erythropoietin (EPO) is a 166 amino acid residues long polypeptide, which is the primary mediator of hypoxic induction of erythropoiesis, which in adulthood is produced by the kidney (+80%). Hypoxia induces an increase in EPO production, which then circulates in the plasma and binds to receptors expressed on erythroid progenitor cells, leading to terminal differentiation of such precursors and increase in red blood mass.

Although human recombinant EPO is a weak immunogen, its repetitive use in, for instance, renal insufficiency, subtle differences in glycosylation or in the preparation procedure can lead to the development of specific neutralizing antibodies. As EPO is the sole mediator of erythropoiesis due to hypoxia, the presence of a neutralizing immune response is considered as a dramatic event.

One way to prevent occurrence of such unwanted immune response is to vaccinate individuals in need for EPO with a promiscuous class II restricted T cell epitope linked to a thioreductase motif located within the epitope flanking region. This elicits epitope-specific CD4+ T cells with cytolytic properties. Administration of a molecule of EPO coupled to the same promiscuous epitope activates cytolytic CD4+ T cells, which eliminate by apoptosis antigen-presenting cells presenting EPO and thereby the capacity to mount an immune response to EPO.

The invariant chain contains a CLIP sequence which is a promiscuous T cell epitope, which binds to nascent MHC class II molecules with a relatively low affinity. CLIP is released from its class II binding by competition with peptides showing higher affinity. During this exchange of epitopes, the DM molecule protects the first anchoring pocket of class II molecules in a transient status between CLIP and the new peptide.

A mutated version of CLIP in which the first 2 amino acids, which show weak affinity for class II binding, are replaced by two hydrophobic residues will maintain its promiscuity while increasing affinity for class II molecules. In addition, the residue located in position 6 can be mutated from P to R to further increase its binding affinity.

Thus, the sequence KM₁R₂MATP₆LLMQAL [SEQ ID NO:9], in which the second and third amino acid (M and R, respectively) are located in position 1 and 2, respectively, are mutated, as well as P in position 6 to give KF₁F₂MATR₆LLMQAL [SEQ ID NO:17].

A peptide is generated by addition of a thioreductase motif and a Val Arg linker sequence at the N terminal, giving the full sequence: CPYC-VR-FFMATRLLMQAL [SEQ ID NO:18].

Patients in need for EPO injection are vaccinated by administration of peptide [SEQ ID NO:18] using a standard procedure of peptide adsorbed on aluminum hydroxide and administered by SC injection. This procedure is known to elicit epitope-specific cytolytic CD4+ T cells, as described in patent application WO200817517.

The sequence KFFMATRLLMQAL [SEQ ID NO:17] is added at the terminal end of EPO, separated by two glycines from the EPO sequence, making a total of 181 amino acids. This modified EPO [SEQ ID NO:19] retains its full activity upon administration and activates the cytolytic CD4+ T cells obtained by vaccination, thereby precluding any detrimental immune response.

The promiscuous nature of the CLIP-modified epitope makes it possible to use the same vaccine and the same EPO molecule for any patient in need of such therapy.

### Example 3: Alpha-Galactosidase

Fabry disease is lysosomal storage disease with accumulation of glycosphingolipids in various tissues due to absence of alpha-galactosidase, a lysosomal hydrolase. It is a X-linked gene defect disease affecting±1 out 100,000 individuals. Current therapy of Fabry disease includes regular infusion of alpha-galactosidase. However, more than 25% of patients under such a therapy develop an immune response to the enzyme, preventing any further use of such enzyme and precipitating patients into risks of various complications, including stroke.

Recombinant alpha-galactosidase can be modified as to contain the sequence of a class II-restricted promiscuous T cell epitope added at the amino-terminal end of the molecule. Administration of this modified alpha-galactosidase molecule to individuals previously vaccinated to this promiscuous epitope containing a thioreductase motif, thereby eliciting the production of peptide-specific cytolytic CD4+ T cells, does not elicit an immune response to alpha-galactosidase.

A promiscuous epitope of apolipoprotein B-100 of sequence LFLKSDGRVKYTLN [SEQ ID NO:20] corresponding to amino acids 1277 to 1290, in which P1 is occupied by L1279 (underlined) is produced by chemical synthesis together with a thioreductase motif, leading to sequence

[SEQ ID NO:21]
CPYC-LF-LKSDGRVKYTLN

Such sequence contains 1 arginine (R) at position P6. This R residue is replaced with citrulline, a amino acid obtained by the action of peptidyl arginine deiminase. This modification results in the loss of a positive charge leading to a higher interaction with MHC class II anchoring residues at P6. The final sequence of the peptide used for vaccination is therefore CPYC-LF-LKSDG-citrulline-VKYTLN [SEQ ID NO:22].

Patients affected by Fabry disease are immunized with peptide of SEQ ID NO:21, using a standard procedure of peptide adsorbed on aluminium hydroxide and administered by SC injection. Epitope-specific cytolytic CD4+ T cells are then produced.

Such vaccinated patients can then be administered with recombinant alpha-galactosidase modified as to contain the LFLKSDGRVKYTLN sequence [SEQ ID NO:20] of the promiscuous epitope [SEQ ID NO:23].

### Example 4: Cell Therapy

Patients under immunosuppressive therapy could benefit from administration of a therapeutic antibody, yet active vaccination using peptides encompassing class II restricted epitopes could be difficult under such circumstances.

However, it is possible to collect cells from peripheral blood of such patients, prepare naïve CD4+ T cells for in vitro transformation into cytolytic cells, which can then be re-administered to the patient in a strictly autologous manner. By doing so, the patient is immediately protected towards unwanted immune responses to the therapeutic agent considered. One representative example of such situation is multiple sclerosis, with patients under immunosuppressive therapy, who could benefit from the administration of antibodies such as the anti-CD52 specific antibody (Campath-1H, Alemtuzumab).

A fifty ml sample of peripheral blood is collected from such patients and naïve CD4+ T cells are prepared by magnetic bead adsorption. Dendritic cells are derived from the monocytes obtained from the same blood sampling, using methods known in the art.

A promiscuous class II-restricted epitope of Mycobacterium cell entry protein, Mce2, DPIELNATLSAVA [SEQ ID NO:24] (amino acids 163 to 175) was chosen (Panigada (2002) Infect. Immun. 70, 79-85).

A thioreductase motif was added at the amino-terminal end of this peptide to generate the sequence CPYC-DP-IELNATLSAVA [SEQ ID NO:25]

Dendritic cells are loaded with peptide of SEQ ID NO:25 and naïve CD4+ T cells are stimulated four times for 7 days with these dendritic cells to generate cytolytic CD4+ T cells.

5×10⁶ cytolytic cells are administered by the IV route to the cell donor.

An anti-CD52 specific antibody was obtained by genetic engineering, which contain the sequence DPIELNATLSAVA [SEQ ID NO:24] added to the amino-terminal end of the molecule, with 2 glycine residues as a linker [SEQ ID NO:26].

Administration of such modified anti-CD52 antibody to an individual having received autologous CD4+ T cells activated in vitro with peptide of SEQ ID NO:25 results in activation of cytolytic CD4+ T cells to the peptide DPIELNATLSAVA [SEQ ID NO:24], thereby preventing any possibility to elicit an immune response towards the therapeutic antibody.

### Example 5: Vector for Expression of Protein in Fusion with Modified CLIP Promiscuous Epitope

The mammalian expression vector pCMV with cytomegalovirus promotor is engineered to allow the expression in CHO (Chinese hamster ovary) cells of any protein for replacement therapy, in fusion with the promiscuous CLIP-derived epitope described in example 2 [SEQ ID NO:17]. Patients in need for injection with a protein for replacement therapy are first vaccinated by administration of a peptide comprising a thioreductase motif and the modified CLIP-derived epitope described in example 2 [SEQ ID NO:18]. This immunization is known to elicit epitope-specific cytolytic CD4+ T cells, as described in patent application WO200817517.

The therapeutic protein of interest, in the form of a fusion protein, flanked by the CLIP sequence retains its full activity upon administration and activates the cytolytic CD4+ T cells obtained by vaccination, thereby precluding any detrimental immune response. The promiscuous nature of the CLIP-modified epitope makes it possible to use the same vaccine and the same EPO molecule for any patient in need of such therapy, regardless of its HLA profile.

To obtain this expression vector, an adaptor was engineered consisting of CLIP-derived epitope preceded by a linker made of 2 glycines and surrounded by restriction enzyme specific sequences Xho-I/Nhe-I as shown below:

| XhoI(*) | |
|---|---|
| GCA CGG CTC GAG GGC GGA AAG TTT TTC ATG GCC ACC | |
| G G K F F M A T | |
| CGT GCC GAG CTC CCG CCT TTC AAA AAG TAC CGG TGG | |
| 10 20 30 | |
| NheI(*) | |
| AGA CTG CTG ATG CAG GCG CTG AGC TAG CTA GTT C | SEQ ID NO: 38 |
| R L L M Q A L S * | SEQ ID NO: 39 |
| TCT GAC GAC TAC GTC CGC GAC TCG ATC GAT CAA G | |
| 40 50 60 | |

By cloning the adaptor into the commercially available expression vector pCMV, modified expression pCMV-CLIP is created with a multiple cloning site for the insertion of Erythropoietin (EPO) cDNA. For this purpose, the sequence coding for EPO was amplified by PCR with a forward primer consisting of the EPO specific sequence and Age-I specific sequence preceded by a KOZAK sequence, and a reverse primer made of EPO specific sequence and Sal-I specific sequence. After digestion Age-I/Sal-I, the EPO construct is inserted into pCMV-CLIP, pre-digested by Age-I and Xho-I. The plasmid is transformed and amplified in DH5-alpha E. coli. After purification and linearization, the expression vector for EPO-CLIP fusion protein is transfected into CHO cells. Then transfected CHO cells were selected on ampicillin and the clone producing the higher level of EPO-CLIP was selected for mass production of the recombinant fusion protein with SEQ ID NO: 40 (modified CLIP sequence underlined)

[SEQ ID NO: 40]
MGVHECPAWL WLLLSLLSLP LGLPVLGAPP RLICDSRVLE
RYLLEAKEAE NITTGCAEHC SLNENITVPD TKVNFYAWKR
MEVGQQAVEV WQGLALLSEA VLRGQALLVN SSQPWEPLQL
HVDKAVSGLR SLTTLLRALR AQKEAISPPD AASAAPLRTI
TADTFRKLFR VYSNFLRGKL KLYTGEACRT GDRVEGGKFF
MATRLLMQAL S

### Example 6: Sequences Disclosed in the Application

| SEQ | |
|---|---|
| ID | |
| NO: | Sequence |
| 1 | [FWYHT]-X(2)-[VILM]-X(2)-[FWYHT] |
| 2 | C-X(2)-C |
| 3 | C-X(2)-S |
| 4 | C-X(2)-T |
| 5 | S-X(2)-C |
| 6 | T-X(2)-C |
| 7 | [CST]-X(2)-C |
| 8 | C-X(2)-[CST] |
| 9 | KMRMATPLLMQAL |
| 10 | [VILMFWYH][RVILMFWYH]MAT[PR]LLM |
| 11 | aKXVAAWTLKAAaZC (a = D-Alanine, X = l-cyclohexylalanine, Z = |
| | aminocaproic acid) |
| 12 | QGYK VLVLNPSVAA T |
| 13 | TTVRLRA YMNTPGLPV |
| 14 | CPYCVRVLVLNPSVAA |
| 15 | CPYCVRYMNTPGLPV |
| 16 | QGYKVLVLNP SVAATTTVRL RAYMNTPGLP VQVQLQQPGA ELVKPGASVK MSCKASGYTF |
| | TSYNMHWVKQ TPGRGLEWIG AIYPGNGDTS YNQKFKGKAT LTADKSSSTA YMQLSSLTSE |
| | DSAVYYCARS TYYGGDWYFN VWGAGTTVTV SAASTKGPSV FPLAPSSKST SGGTAALGCL |
| | VKDYFPEPVT VSWNSGALTS GVHTFPAVLQ SSGLYSLSSV VTVPSSSLGT QTYICNVNHK |
| | PSNTKVDKKV EPKSCDKTHT CPPCPAPELL GGPSVFLFPP KPKDTLMISR TPEVTCVVVD |
| | VSHEDPEVKF NWYVDGVEVH NAKTKPREEQ YNSTYRVVSV LTVLHQDWLN GKEYKCKVSN |
| | KALPAPIEKT ISKAKGQPRE PQVYTLPPSR DELTKNQVSL TCLVKGFYPS DIAVEWESNG |
| | QPENNYKTTP PVLDSDGSFF LYSKLTVDKS RWQQGNVFSC SVMHEALHNH YTQKSLSLSP GK |
| 17 | KFFMATRLLMQAL |
| 18 | CPYCVRFFMATRLLMQAL |
| 19 | APPRLICDSR VLERYLLEAK EAENITTGCA EHCSLNENIT VPDTKVNFYA WKRMEVGQQA |
| | VEVWQGLALL SEAVLRGQAL LVNSSQPWEP LQLHVDKAVS GLRSLTTLLR ALGAQKEAIS |
| | PPDAASAAPL RTITADTFRK LFRVYSNFLR GKLKLYTGEA CRTGDRGGKF FMATRLLMQA L |
| 20 | LFLKSDGRVKYTLN |
| 21 | CPYCLFLKSDGRVKYTLN |
| 22 | CPYCLFLKSDG-citrulline-VKYTLN |
| 23 | LFLKSDGRVK YTLNDNGLAR TPTMGWLHWE RFMCNLDCQE EPDSCISEKL FMEMAELMVS |
| | EGWKDAGYEY LCIDDCWMAP QRDSEGRLQA DPQRFPHGIR QLANYVHSKG LKLGIYADVG |
| | NKTCAGFPGS FGYYDIDAQT FADWGVDLLK FDGCYCDSLE NLADGYKHMS LALNRTGRSI |
| | VYSCEWPLYM WPFQKPNYTE IRQYCNHWRN FADIDDSWKS IKSILDWTSF NQERIVDVAG |
| | PGGWNDPDML VIGNFGLSWN QQVTQMALWA IMAAPLFMSN DLRHISPQAK ALLQDKDVIA |
| | INQDPLGKQG YQLRQGDNFE VWERPLSGLA WAVAMINRQE IGGPRSYTIA VASLGKGVAC |
| | NPACFITQLL PVKRKLGFYE WTSRLRSHIN PTGTVLLQLE NTMQMSLKDL L |
| 24 | DPIELNATLSAVA |
| 25 | CPYCDPIELNATLSAVA |
| 26 | DPIELNATLS AVAGGQVQLQ ESGPGLVRPS QTLSLTCTVS GFTFTDFYMN WVRQPPGRGL |
| | EWIGFIRDKA KGYTTEYNPS VKGRVTMLVD TSKNQFSLRL SSVTAADTAV YYCAREGHTA |
| | APFDYWGQGS LVTVSSASTK GPSVFPLAPS SKSTSGGTAA LGCLVKDYFP EPVTVSWNSG |
| | ALTSGVHTFP AVLQSSGLYS LSSVVTVPSS SLGTQTYICN VNHKPSNTKV DKKVEPKSCD |
| | KTHTCPPCPA PELLGGPSVF LFPPKPKDTL MISRTPEVTC VVVDVSHEDP EVKFNWYVDG |
| | VEVHNAKTKP REEQYNSTYR VVSVLTVLHQ DWLNGKEYKC KVSNKALPAP IEKTISKAKG |
| | QPREPQVYTL PPSRDELTKN QVSLTCLVKG FYPSDIAVEW ESNGQPENNY KTTPPVLDSD |
| | GSFFLYSKLT VDKSRWQQGN VFSCSVMHEA LHNHYTQKSL SLSPGK |
| 27 | [FWYH]-X(2)-[VILM]-X(2)-[FWYH] |
| 28 | [FWY]-X(2)-[VILM]-X(2)-[FWY] |
| 29 | X1X2MATX6LLM [SEQ ID NO: 29], X1 and X2 are V, I, L, M, F, H, Y or W, |
| | X6 is R or P. |
| 30 | FFMATRLLM |
| 31 | WWMATRLLM |
| 32 | WFMATRLLM |
| 33 | FWMATRLLM |
| 34 | FFMATPLLM |
| 35 | WWMATPLLM |
| 35 | WFMATPLLM |
| 37 | FWMATPLLM |
| 38 | gca cgg ctc gag ggc gga aag ttt ttc atg gcc acc aga ctg ctg atg cag |
| | gcg ctg agc tag cta gtt c |
| 39 | GGKFFMATRLLMQALS |
| 40 | MGVHECPAWL WLLLSLLSLP LGLPVLGAPP RLICDSRVLE RYLLEAKEAE NITTGCAEHC |
| | SLNENITVPD TKVNFYAWKR MEVGQQAVEV WQGLALLSEA VLRGQALLVN SSQPWEPLQL |
| | HVDKAVSGLR SLTTLLRALR AQKEAISPPD AASAAPLRTI TADTFRKLFR VYSNFLRGKL |
| | KLYTGEACRT GDRVEGGKFF MATRLLMQAL S |

1-2. (canceled)

3. A kit of parts of comprising:
a) a peptide comprising:
a1) a first peptide sequence comprising an MHC class II T cell epitope or a CD1d-restricted NKT cell epitope, and
a2) immediately adjacent to said first peptide sequence or separated by at most 7 amino acids from said first peptide sequence a [CST]-X(2)-C [SEQ ID NO:7] or C-X(2)-[CST] [SEQ ID NO:8] oxidoreductase motif sequence,
and
b) a viral vector for gene therapy or gene vaccination comprising a fusion protein comprising:
b1) a viral vector protein for the function and maintenance of the vector, and
b2) the first peptide sequence defined in a1), wherein the first peptide sequence does not occur in the sequence of the protein of b1).

4. (canceled)

5. The kit of parts according to claim 3, wherein the oxidoreductase motif sequence is C-X(2)-C [SEQ ID NO:2].

6. The kit of parts according to claim 3, wherein the CD1d-restricted NKT cell epitope motif has the sequence [FWYHT]-X(2)-[VILM]-X(2)-[FWYHT] [SEQ ID NO:1].

7. The kit of parts according to claim 3, wherein the CD1d-restricted NKT cell epitope motif has the sequence [FWY]-X(2)-[VILM]-X(2)-[FWY] [SEQ ID NO:28].

8. The kit of parts according to claim 3, wherein said MHC class II T cell epitope is a promiscuous epitope binding to one or more HLA-DR1 molecules.

9. The kit of parts according to claim 3, wherein said MHC class II T cell epitope has the sequence X₁X₂MATX₆LLM [SEQ ID NO:29], wherein X₁ and X₂ are independently selected from V, I, L, M, Y, H, F and W, and X₆ is R or P.

10-12. (canceled)

13. A method for gene therapy or gene vaccination comprising administering to a subject in need thereof an effective amount of a viral vector comprising a fusion protein comprising:
b1) a viral protein for the function and maintenance of the vector, and
b2) a first peptide sequence comprising an MHC class II T cell epitope or a CD1d-restricted NKT cell epitope, wherein the first peptide sequence is a sequence which does not occur in the sequence of the protein of b1,
wherein the subject has been previously administered an effective amount of a peptide comprising:
a1) said first peptide sequence and
a2) immediately adjacent to said first peptide sequence or separated by at most 7 amino acids from said first peptide sequence a sequence with a [CST]-X(2)-C [SEQ ID NO:7] or C-X(2)-[CST] [SEQ ID NO:8] oxidoreductase motif sequence

14-15. (canceled)

16. A method for preparing a kit of parts, comprising:
a) preparing a viral vector comprising a modified viral vector protein for the function and maintenance of the vector by introducing into the sequence of said protein the sequence of an MHC class II T cell epitope or a CD1d-restricted NKT cell peptide epitope, which epitope sequence is a sequence which does not occur in the unmodified protein,
b) preparing a peptide comprising:
the MHC class II T cell epitope or a CD1d-restricted NKT cell peptide epitope of a), and
immediately adjacent to said epitope or separated by at most 7 amino acids from said epitope a [CST]-X(2)-C [SEQ ID NO:7] or C-X(2)-[CST] [SEQ ID NO:8] oxidoreductase motif sequence.

17-24. (canceled)

25. The method according to claim 13, wherein the oxidoreductase motif sequence is C-X(2)-C [SEQ ID NO:2].

26. The method according to claim 13, wherein said MHC class II T cell epitope is a promiscuous epitope binding to one or more HLA-DR1 molecules.

27. The method according to claim 13, wherein said MHC class II T cell epitope has the sequence X₁X₂MATX₆LLM [SEQ ID NO:29], wherein X₁ and X₂ are independently selected from V, I, L, M, Y, H, F and W, and X₆ is R or P.

28. The method according to claim 16, wherein the oxidoreductase motif sequence is C-X(2)-C [SEQ ID NO:2].

29. The method according to claim 16, wherein said MHC class II T cell epitope is a promiscuous epitope binding to one or more HLA-DR1 molecules.

30. The method according to claim 16, wherein said MHC class II T cell epitope has the sequence X₁X₂MATX₆LLM [SEQ ID NO:29], wherein X₁ and X₂ are independently selected from V, I, L, M, Y, H, F and W, and X₆ is R or P.

31. A method of gene therapy or gene vaccination, comprising administering to an individual in need thereof
a) an effective amount of the peptide of claim 3 a); and
b) the vector of claim 3 b).

32. The method according to claim 31, wherein the oxidoreductase motif sequence is C-X(2)-C [SEQ ID NO:2].

33. The method according to claim 31, wherein said MHC class II T cell epitope is a promiscuous epitope binding to one or more HLA-DR1 molecules.

34. The method according to claim 31, wherein said MHC class II T cell epitope has the sequence X₁X₂MATX₆LLM [SEQ ID NO:29], wherein X₁ and X₂ are independently selected from V, I, L, M, Y, H, F and W, and X₆ is R or P.


SEQUENCE LISTING (40 sequences)

SEQ ID NO: 1-8 - skipped (8 entries)

SEQ ID NO: 9 - protein, 13 aa, synthetic construct
  REGION 1..13: note = CLIP peptide
KMRMATPLLMQAL

SEQ ID NO: 10 - protein, 9 aa, synthetic construct
  REGION 1..9: note = modified clip
  VARIANT 1: note = Xaa is Val, Ile, Leu, Met, Phe, Trp, Tyr or His
  VARIANT 2: note = Xaa is Arg, Val, Ile, Leu, Met, Phe, Trp, Tyr or His
  VARIANT 6: note = Xaa is Pro or Arg
XXMATXLLM

SEQ ID NO: 11 - protein, 15 aa, synthetic construct
  REGION 1..15: note = Padre peptide
  MOD_RES 1: note = D_alanine
  MOD_RES 3: note = I-cyclohexylalanine
  MOD_RES 13: note = D_alanine
  MOD_RES 14: note = alpha aminocaproic acid
AKAVAAWTLKAAAKC

SEQ ID NO: 12 - protein, 15 aa, synthetic construct
  REGION 1..15: note = hepatitis C fragment 1247-1261
QGYKVLVLNPSVAAT

SEQ ID NO: 13 - protein, 16 aa, synthetic construct
  REGION 1..16: note = hepatitis C fragment 1535-1550
TTVRLRAYMNTPGLPV

SEQ ID NO: 14 - protein, 16 aa, synthetic construct
  REGION 1..16: note = CxxC redox motif +hepatitis C epitope
CPYCVRVLVLNPSVAA

SEQ ID NO: 15 - protein, 15 aa, synthetic construct
  REGION 1..15: note = CxxC redox motif +hepatitis C epitope
CPYCVRYMNTPGLPV

SEQ ID NO: 16 - protein, 482 aa, synthetic construct
  REGION 1..482: note = fusion protein of hepatitis c fragment and anti-CD20 antibody
QGYKVLVLNPSVAATTTVRLRAYMNTPGLPVQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 13 aa, synthetic construct
  REGION 1..13: note = modified clip peptide
KFFMATRLLMQAL

SEQ ID NO: 18 - protein, 18 aa, synthetic construct
  REGION 1..18: note = CxxC redox motif +modified clip peptide
CPYCVRFFMATRLLMQAL

SEQ ID NO: 19 - protein, 181 aa, synthetic construct
  REGION 1..181: note = EPO +modified clip peptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGKFFMATRLLMQAL

SEQ ID NO: 20 - protein, 14 aa, synthetic construct
  REGION 1..14: note = apolipoprotein B-100 peptide sequence
LFLKSDGRVKYTLN

SEQ ID NO: 21 - protein, 18 aa, synthetic construct
  REGION 1..18: note = CxxC +redox motif apolipoprotein B-100 epitope
CPYCLFLKSDGRVKYTLN

SEQ ID NO: 22 - protein, 18 aa, synthetic construct
  REGION 1..18: note = citrulinnated version of SEQ IDN O 21
  MOD_RES 12: note = citrulinne
CPYCLFLKSDGRVKYTLN

SEQ ID NO: 23 - protein, 411 aa, synthetic construct
  REGION 1..411: note = apolipoprotein B-100 epitope +alpha galactosidase
LFLKSDGRVKYTLNDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 24 - protein, 13 aa, synthetic construct
  REGION 1..13: note = mycobacterium cell entry protein
DPIELNATLSAVA

SEQ ID NO: 25 - protein, 17 aa, synthetic construct
  REGION 1..17: note = redox motif +mycobacterium cell entry protein epitope
CPYCDPIELNATLSAVA

SEQ ID NO: 26 - protein, 466 aa, synthetic construct
  REGION 1..466: note = mycobacterium cell entry protein +anti-CD52 specific antibody
DPIELNATLSAVAGGQVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27-28 - skipped (2 entries)

SEQ ID NO: 29 - protein, 9 aa, synthetic construct
  REGION 1..9: note = modified clip peptide
  VARIANT 1..2: note = Xaa is Val, Ile, Leu, Met, Tyr, His, Phe or Trp
  VARIANT 6: note = Xaa is Arg or Pro
XXMATXLLM

SEQ ID NO: 30 - protein, 9 aa, synthetic construct
  REGION 1..9: note = modified clip peptide
FFMATRLLM

SEQ ID NO: 31 - protein, 9 aa, synthetic construct
  REGION 1..9: note = modified clip peptide
WWMATRLLM

SEQ ID NO: 32 - protein, 9 aa, synthetic construct
  REGION 1..9: note = modified clip peptide
WFMATRLLM

SEQ ID NO: 33 - protein, 9 aa, synthetic construct
  REGION 1..9: note = modified clip peptide
FWMATRLLM

SEQ ID NO: 34 - protein, 9 aa, synthetic construct
  REGION 1..9: note = modified clip peptide
FFMATPLLM

SEQ ID NO: 35 - protein, 9 aa, synthetic construct
  REGION 1..9: note = modified clip peptide
WWMATPLLM

SEQ ID NO: 36 - protein, 9 aa, synthetic construct
  REGION 1..9: note = modified clip peptide
WFMATPLLM

SEQ ID NO: 37 - protein, 9 aa, synthetic construct
  REGION 1..9: note = modified clip peptide
FWMATPLLM

SEQ ID NO: 38 - DNA, 70 bp, synthetic construct
  misc_feature 1..70: note = fragment of DNA vector for cloning fusion proteins with promiscuous epitope
  source 1..70: mol_type = other DNA
gcacggctcgagggcggaaagtttttcatggccaccagactgctgatgcaggcgctgagctagctagttc

SEQ ID NO: 39 - protein, 16 aa, synthetic construct
  REGION 1..16: note = promiscuous MHC class II T cell epitope
GGKFFMATRLLMQALS

SEQ ID NO: 40 - protein, 211 aa, synthetic construct
  REGION 1..211: note = fusion protein of erythropoeitein and promiscuous MHC Class II epitope
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALRAQKEAISPPDAASAAPLRTITADTFRKLFRVYSNFLRGKLKLYTGEACRTGDRVEGGKFFMATRLLMQALS